(19) United States
(12) Patent Application Publication
(10) Pub. No.: US 2024/0374658 A1
(43) Pub. Date: Nov. 14, 2024

(54) COMPOSITION FOR PREVENTING, TREATING OR ALLEVIATING RHEUMATOID ARTHRITIS COMPRISING PROPIONIBACTERIUM FREUDENREICHII MJ2 STRAIN AS AN ACTIVE INGREDIENT

(71) Applicant: KOREA UNIVERSITY RESEARCH AND BUSINESS FOUNDATION, Seoul (KR)

(72) Inventors: Young Hee LIM, Seoul (KR); Jiah YEOM, Seoul (KR); Seong-ho MA, Seoul (KR); Dong-Joon YIM, Seoul (KR)

(73) Assignee: KOREA UNIVERSITY RESEARCH AND BUSINESS FOUNDATION, Seoul (KR)

(21) Appl. No.: 18/701,173

(22) PCT Filed: Jan. 24, 2022
(86) PCT No.: PCT/KR2022/001189

§ 371 Date: Apr. 12, 2024

(30) Foreign Application Priority Data
Jan. 25, 2021 (KR) 10-2021-0010296

(51) Int. Cl. A61K 35/741; A61P 19/02; C12N 1/20

(52) U.S. Cl. CPC A61K 35/741; A61P 19/02; C12N 1/20

(57) ABSTRACT

The present invention relates to a composition including a Propionibacterium freudenreichii MJ2 strain as an active ingredient for preventing, alleviating, or treating rheumatoid arthritis. The composition of the present invention is safe even when administered for a long period of time and restores autophagy to a normal level while inhibiting osteoclast differentiation, inflammatory reactions, apoptosis, and antibody production, and is thus expected to be variously used for drugs, quasi-drugs, functional foods, and the like for preventing, treating, and alleviating rheumatoid arthritis, which is an autoimmune disease.

### CROSS REFERENCE TO RELATED APPLICATIONS

This application is a National Stage of International Application No. PCT/KR2022/001189 filed Jan. 24, 2022, claiming priority based on Korean Patent Application No. 10-2021-0010296 filed Jan. 25, 2021, the entire disclosures of which are incorporated herein by reference.

### BACKGROUND ART

Rheumatoid arthritis is a chronic systemic inflammatory autoimmune disease that is accompanied by swelling and pain due to continuous inflammatory reactions in the joint synovial membrane, and causes destruction of joint cartilage and bone damage. Typical early symptoms include swelling and pain in the joints of the hands and feet, and joints becoming stiff in the morning to make movement difficult. In addition, it is known to be accompanied by symptoms other than arthritis, such as osteoporosis, vasculitis, and organ involvement.

To date, a cure drug for rheumatoid arthritis has not been developed, but a non-steroidal anti-inflammatory drug, an immunosuppressant, an anti-rheumatic drug, and the like are used as a therapeutic agent for rheumatoid arthritis. However, although the non-steroidal anti-inflammatory drug is effective in relieving pain, the non-steroidal anti-inflammatory drug does not prevent the progression of the disease and may cause gastrointestinal disorders when taken for a long period of time, and the immunosuppressant such as a TNF-α inhibitor may cause side effects such as latent tuberculosis.

Therefore, there is a need to develop a novel therapeutic drug that may replace these drugs and has fewer side effects while suppressing the symptoms of rheumatoid arthritis.

Recently, in addition to various effects of lactic acid bacteria, it has been found that lactic acid bacteria are effective in suppressing rheumatoid arthritis. In a model where arthritis is induced with collagen, consumption of Lactobacillus helveticus was effective in suppressing arthritis, and L. reuteri, L. casei, and L. rhamnosus also showed effects in alleviating arthritis. In this regard, research is continuing to discover therapeutic agents for rheumatoid arthritis using microorganisms.

### PRIOR ARTS

### Non-Patent Document

(Non-Patent Document 0001) Food & Function, 2020; 11(4), 3681-3694
(Non-Patent Document 0002) Journal of functional foods, 2015; 13, 350-362

### DISCLOSURE

### Technical Problem

An object of the present invention is to provide a composition capable of preventing, alleviating, or treating rheumatoid arthritis.

However, technical objects of the present invention are not limited to the aforementioned purpose and other objects which are not mentioned may be clearly understood to those skilled in the art from the following description.

### Technical Solution

In order to solve the above object, an aspect of the present invention provides a pharmaceutical composition for preventing or treating rheumatoid arthritis comprising Propionibacterium freudenreichii MJ2 (KCCM12272P), a culture solution thereof, live cells thereof, dead cells thereof, or a mixture thereof as an active ingredient.

Another aspect of the present invention provides a food composition for preventing or alleviating rheumatoid arthritis containing Propionibacterium freudenreichii MJ2, a culture solution thereof, live cells thereof, dead cells thereof, or a mixture thereof as an active ingredient.

In an embodiment of the present invention, the live cells may be obtained by culturing the MJ2 strain activated in a culture medium for 45 to 50 hours under anaerobic conditions at 30 to 37° C. and centrifuging the cultured strain solution.

In another embodiment of the present invention, the dead cells may be obtained by culturing the MJ2 strain activated in a culture medium for 45 to 50 hours under anaerobic conditions at 30 to 37° C. and heat-treating the cultured strain.

In yet another embodiment of the present invention, the composition may inhibit inflammatory reactions by increasing the expression of IL-10.

In yet another embodiment of the present invention, the composition may inhibit osteoclast differentiation.

In yet another embodiment of the present invention, the composition may decrease production of IgG, IgG1, and IgG2a.

In yet another embodiment of the present invention, the composition may inhibit apoptosis and restore autophagy to a normal level.

In yet another embodiment of the present invention, the composition may include MJ2 live cells at a concentration of 1×10⁶ to 1×10¹⁰ CFU/mL, preferably 1×10⁷ to 1×10⁹ CFU/mL, and more preferably 1×10⁸ CFU/mL.

In yet another embodiment of the present invention, the composition may include MJ2 dead cells at a concentration of 1×10⁶ to 1×10¹⁰ cells/mL, preferably 1×10⁷ to 1×10⁹ cells/mL, and more preferably 1×10⁸ cells/mL.

In yet another embodiment of the present invention, the composition may be a mixture of MJ2 live cells and dead cells.

In yet another embodiment of the present invention, the composition may be administered once a day and may be for oral administration.

Another aspect of the present invention provides a method for alleviating, preventing, or treating rheumatoid arthritis comprising administering Propionibacterium freudenreichii MJ2, a culture solution thereof, live cells thereof, dead cells thereof, or a mixture thereof to a subject. The administration may be oral administration, and the method may further include checking the degree of swelling of the joint after the administration step.

In an embodiment of the present invention, the method may comprise administering MJ2 live cells and/or dead cells to the subject, and the MJ2 live cells and/or dead cells may be administered at 10⁶ to 10¹⁰ cells/day, preferably 10⁷ to 10⁹ cells/day, and more preferably 10⁸ cells/day.

Yet another aspect of the present invention provides a use of Propionibacterium freudenreichii MJ2, a culture solution thereof, live cells thereof, dead cells thereof, or a mixture thereof for preparing a drug for preventing or treating rheumatoid arthritis.

### Advantageous Effects

According to the present invention, the composition is safe even when administered for a long period of time, restores osteoclast differentiation, inflammatory reactions, apoptosis, and autophagy to normal levels, and inhibits antibody production, and is thus expected to be variously used for drugs, quasi-drugs, functional foods and the like for preventing, treating, and alleviating rheumatoid arthritis, which is an autoimmune disease.

### DESCRIPTION OF DRAWINGS

FIG. 1 is a result illustrating expression inhibition of inflammatory cytokines by a strain of the present invention.

FIG. 2 is a result illustrating expression inhibition of osteoclast differentiation factors by the strain of the present invention.

FIG. 3 is a result illustrating inhibition of TRAP activity in osteoclasts by the strain of the present invention.

FIG. 4 is a result illustrating increased expression of an anti-inflammatory cytokine by the strain of the present invention.

FIG. 5 is a result illustrating arthritis disease scores in a mouse model administered orally with the strain of the present invention.

FIGS. 6A and 6B show results illustrating micro-CT images (FIG. 6A) and scores (FIG. 6B) in a mouse model administered orally with the strain of the present invention.

FIG. 7 is a result of measuring collagen antigen-specific IgG, IgG1, and IgG2a antibodies in a mouse model administered orally with the strain of the present invention.

FIG. 8 is a result of measuring index of spleen, expression levels of inflammatory cytokines and an anti-inflammatory cytokine in a mouse model administered orally with the strain of the present invention.

FIG. 9 is a result of measuring expression levels of osteoclast differentiation genes in a mouse model administered orally with the strain of the present invention.

FIG. 10 is a result of measuring expression levels of apoptosis proteins in a mouse model administered orally with the strain of the present invention.

FIG. 11 is a result of measuring expression levels of autophagy proteins in a mouse model administered orally with the strain of the present invention.

### MODES OF THE INVENTION

Propionibacterium freudenreichii was first identified in cheese fermentation studies and may mainly be found in milk and dairy products. Recently, it has been found that P. freudenreichii is effective in treating colitis by adhering to the surface of the large intestine to control cytokine release.

The P. freudenreichii used by the present inventors is a P. freudenreichii MJ2 strain (KCCM12272P) deposited at the Korean Culture Center of Microorganisms.

The present inventors confirmed that the P. freudenreichii MJ2 strain has an effect of increasing the expression of anti-inflammatory cytokine IL-10 in vitro and simultaneously inhibiting osteoclast differentiation, confirmed that the MJ2 strain may prevent rheumatoid arthritis in a collagen-induced animal model, and then completed the present invention.

Specifically, the present inventors induced inflammation by treating Raw 264.7 macrophages with LPS. As a result of treating P. freudenreichii MJ2 dead cells with LPS at various concentrations and checking the expression levels of inflammatory cytokines IL-6, TNF-α, IL-1p, and MMP9, a significant decrease in cytokine expression was confirmed in a group treated with 1×10⁷ cells/mL of MJ2 dead cells (Example 2).

In addition, as a result of treating Raw 264.7 macrophage cells with RANKL (receptor activator of nuclear factor kappa-B ligand) to induce differentiation into osteoclasts, and treating P. freudenreichii MJ2 dead cells with RANKL at various concentrations to confirm the expression levels of osteoclast differentiation markers comprising MMP9, RANK, c-fos, NFATc1, Calcr, and Ctsk, the present inventors confirmed that the expression of osteoclast differentiation markers was reduced when treated with MJ2 dead cells at concentrations of 1×10⁶ cells/mL and 1×10⁷ cells/mL. From the result, it was found that MJ2 dead cells had osteoclast differentiation inhibitory activity, and the present inventors reconfirmed the osteoclast differentiation inhibitory activity of MJ2 dead cells using tartrate-resistant acid phosphate (TRAP) staining (Example 3).

In addition, the present inventors confirmed that MJ2 dead cells increased the expression of anti-inflammatory cytokine IL-10 that inhibited inflammatory reactions while inhibiting osteoclast differentiation (Example 4).

Next, the present inventors intended to confirm whether MJ2 dead cells, which inhibited osteoclast differentiation and had anti-inflammatory activity in vitro, may prevent rheumatoid arthritis when included in orally administered drugs or foods. Specifically, it was confirmed that MJ2 live and dead cells were orally administered to mice at low or high concentrations for 3 weeks to induce arthritis with collagen, symptoms of arthritis in the knee joints and paws were observed, and arthritis symptoms were mild in MJ2 live cells and/or dead cells from micro-CT scan image analysis (Examples 5-1 and 5-2).

In addition, as a result of measuring the amount of each IgG in plasma to serologically confirm the severity of symptoms of rheumatoid arthritis, which is an autoimmune disease, the present inventors confirmed that the concentration of collagen antigen-specific total IgG and IgG2a antibodies in the MJ2-administered group was significantly lower than that in a non-administered group (Example 5-3). From the result, it can be seen that MJ2 live and/or dead cells may prevent rheumatoid arthritis.

In addition, a significant decrease in spleen index was confirmed in a group administered with low-concentration MJ2 live or dead cells, a significant decrease in IL-6 expression was confirmed in a group administered with MJ2 dead cells, and a significant decrease in TNF-α and an increase in IL-10 at all concentrations were confirmed in the group administered with MJ2 live or dead cells. In particular, a significant decrease in TNF-α was confirmed in a group treated with high-concentration MJ2 dead cells, and a significant increase in IL-10 was confirmed in a group treated with low-concentration MJ2 live cells (Example 5-4). From the results, it can be seen that the MJ2 live and/or dead cells may inhibit inflammatory reactions to alleviate and treat rheumatoid arthritis.

In addition, it can be seen that the MJ2 live and dead cells may inhibit osteoclast differentiation even in vivo, reduce apoptosis of cells around the synovial membrane, and restore autophagy to a normal level (Examples 5-5 to 5-7). From the result, it can be seen that the MJ2 live and/or dead cells may inhibit the progression of rheumatoid arthritis by increasing the survival rate of cells around the synovial membrane where inflammatory reactions are induced.

Therefore, the present invention provides a pharmaceutical composition for preventing or treating rheumatoid arthritis containing Propionibacterium freudenreichii MJ2, a culture solution thereof, live cells thereof, dead cells thereof, or a mixture thereof as an active ingredient.

The pharmaceutical composition according to the present invention may include a pharmaceutically acceptable carrier in addition to the active ingredient. At this time, the pharmaceutically acceptable carrier is commonly used in formulation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc, but is not limited thereto. Further, the pharmaceutical composition may further include a lubricant, a wetting agent, a sweetening agent, a flavoring agent, an emulsifier, a suspension, a preservative, and the like, in addition to the ingredients.

The pharmaceutical composition of the present invention may be administered orally or parenterally (e.g., applied intravenously, subcutaneously, intraperitoneally, or topically) according to a desired method, and a dose thereof varies depending on the condition and body weight of a patient, the severity of a disease, a drug form, and route and time of administration, but may be appropriately selected by those skilled in the art.

As used herein, the “prevention” means all actions that inhibit rheumatoid arthritis or delay the onset of rheumatoid arthritis by administration of the pharmaceutical composition according to the present invention.

As used herein, the “treatment” means all actions that improve or beneficially change symptoms of rheumatoid arthritis by administration of the pharmaceutical composition according to the present invention.

As used herein, the “alleviating” means all actions that reduce parameters related to rheumatoid arthritis, such as the severity of symptoms by administration of the composition according to the present invention.

The pharmaceutical composition of the present invention is administered in a pharmaceutically effective amount. As used herein, the “pharmaceutically effective amount” refers to an amount enough to treat the disease at a reasonable benefit/risk ratio applicable to medical treatment. The effective dose level may be determined according to factors including the type and severity of a disease of a patient, the activity of a drug, the sensitivity to a drug, a time of administration, a route of administration, an excretion rate, duration of treatment, and simultaneously used drugs, and other factors well-known in the medical field. The pharmaceutical composition according to the present invention may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

Specifically, the effective amount of the pharmaceutical composition of the present invention may vary depending on the age, sex, condition, and body weight of a patient, absorption, inactivation rate, and excretion rate of an active ingredient in the body, a type of disease, and a combined drug, and generally, may be administered at 0.001 to 150 mg, preferably 0.01 to 100 mg per 1 kg of body weight every day or every other day, or separately administered 1 to 3 times per day. However, since the effective amount may increase or decrease depending on the route of administration, severity of rheumatoid arthritis, sex, body weight, age, etc., the dose does not limit the scope of the present invention in any way.

Yet another aspect of the present invention provides a method for preventing, regulating, or treating rheumatoid arthritis, including administering the pharmaceutical composition to a subject.

As used herein, the “subject” refers to a subject in need of the method for preventing, regulating, or treating the disease, and more particularly, refers to mammals, such as human or non-human primates, mice, dogs, cats, horses and cattle.

Yet another aspect of the present invention provides a health functional food composition for alleviating rheumatoid arthritis including P. freudenreichii MJ2, a culture solution thereof, live cells thereof, dead cells thereof, or a mixture thereof as an active ingredient. More specifically, the composition of the present invention may be added to health functional foods for the purpose of preventing or alleviating rheumatoid arthritis, and when using the composition of the present invention as a food additive, the composition may be added as it is or used together with other foods or food ingredients, and may be used appropriately according to conventional methods. The mixed amount of the active ingredients may be suitably determined according to a purpose of use (prevention, health, or therapeutic treatment). In general, when preparing foods or beverages, the compound of the present invention is added in an amount of 15 wt % or less, preferably 10 wt % or less, based on the raw material. However, in the case of long-term intake for the purpose of health and hygiene or for the purpose of health control, the amount may be equal to or lower than the range, and there is no problem in terms of safety, so that the active ingredients may be used even in an amount above the range.

The kind of food is not particularly limited. Examples of the food which may be added with the materials include meat, sausages, bread, chocolate, candies, snacks, confectionery, pizza, ramen, other noodles, gums, dairy products including ice cream, various soups, beverages, tea, drinks, alcohol drinks, vitamin complex, and the like, and include all health functional foods in an accepted meaning.

The health beverage composition of the present invention, like a general beverage, may include various flavoring agents or natural carbohydrates as an additional ingredient. The above-mentioned natural carbohydrates include monosaccharides such as glucose and fructose, disaccharides such as maltose and sucrose, polysaccharides such as dextrin and cyclodextrin, and sugar alcohols such as xylitol, sorbitol and erythritol. As the sweetening agent, natural sweetening agents such as thaumatin and stevia extracts, synthetic sweetening agents such as saccharin and aspartame, and the like may be used. A ratio of the natural carbohydrates may be generally about 0.01 to 0.20 g, preferably about 0.04 to 0.10 g per 100 ml of the composition of the present invention.

The composition of the present invention may contain various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, or the like, in addition to the ingredients. In addition, the composition of the present invention may include pulps for preparing natural fruit juices, fruit juice beverages and vegetable beverages. These ingredients may be used independently or in combination. Although the ratio of these additives is not greatly important, generally, the ratio thereof is selected in a range of 0.01 to 0.20 part by weight per 100 parts by weight of the composition of the present invention.

The present invention may have various modifications and various Examples, and specific Examples will be hereinafter illustrated in the drawings and described in detail in the detailed description. However, this does not limit the present invention within specific embodiments, and it should be understood that the present invention covers all the modifications, equivalents and replacements within the idea and technical scope of the present invention. In describing the present invention, a detailed description of related known technologies will be omitted if it is determined that they make the gist of the present invention unclear.

### EXAMPLES

### Example 1: Preparation of Live Cells and Dead Cells of Propionibacterium freudenreichii MJ2

Propionibacterium freudenreichii MJ2 (KCCM12272P) used in the present invention was a strain deposited at the Korean Culture Center of Microorganisms. P. freudenreichii MJ2 was a strain that was selected as a candidate strain through a process of purifying single colonies three times from raw milk procured from a farm in Gimpo, Gyeonggi-do, and then identified using 16s rDNA sequencing.

To prepare live and dead cells of Propionibacterium freudenreichii MJ2, the activated strain was inoculated in a Reinforced Clostridial Medium (RCM) at least three times, and then incubated at 30° C. for 48 hours in an anaerobic environment created using GasPak (Franklin lakes, NJ, USA). The cultured strain solution was centrifuged at 3,000 rpm to collect live cells, diluted in phosphate-buffered saline (PBS) to 10⁸ CFU/mL, and then heated to prepare dead cells.

### Example 2: Measurement of Inflammatory Cytokine Expression Inhibitory Ability of P. Freudenreichii MJ2 Dead Cells

2-1. Culture of Macrophage and Treatment of P. freudenreichii MJ2 Dead Cells

A Raw 264.7 cell line, a macrophage cell line, was incubated in a 37° C. incubator under a 5% CO₂-95% air composition in a Dulbecco's modified eagle medium (DMEM) added with 10% fetal bovine serum (FBS), 100 units/mL penicillin, and 100 μg/mL streptomycin. When the cells grew to about 80% confluent, the cells were removed using a scraper to be adjusted to 2.5×10⁸ cells/mL, and then 2 mL each was inoculated into 6 wells. The cells were stabilized in a 37° C. incubator for one day, and then treated with 100 ng/mL lipopolysaccharides (LPS) and P. freudenreichii MJ2 dead cells (1×10⁵, 1×10⁶, and 1×10⁷ cells/mL) and cultured for 24 hours.

2-2. Measurement of Inflammatory Cytokine Expression Inhibitory Ability of P. freudenreichii MJ2 Dead Cells

A macrophage culture supernatant cultured by treating LPS and MJ2 dead cells in Example 2-1 was removed, and RNA was isolated using Trizol. The isolated RNA was converted into cDNA using a revert aid first strand cDNA kit (Thermo Fisher Scientific, Waltham, MA, USA), pre-heated at 95° C. for 10 minutes using an Applied Biosystems 7500 (Applied Biosystems, Foster City, CA, USA) machine with a DyNamo HS SYBR Green qPCR kit (FINNZYMES, Finland) reagent, and then subjected to real-time PCR by cycling 40 times at 95° C. for 15 seconds, 60° C. for 15 seconds, and 72° C. for 30 seconds. The sequences of the primers used were shown in Table 1 below.

TABLE 1

| Gene | Forward (5′-3′) | Reverse (5′-3′) |
|---|---|---|
| GAPDH | ACCCAGAAGACTGTGGATGG | CACATTGGGGGTAGGAACAC |
| IL-6 | TTTCTCCACGCAGGAGACTT | TCCACGATTTCCCAGAGAAC |
| TNF-α | AAGATGGAGGAAGGGCAGTT | GATCCTGGAGGGGAAGAGAC |
| IL-1β | CAGGCAGGCAGTATCACTCA | AGCTCATATGGGTCCGACAG |
| MMP9 | GAAGGCAAACCCTGTGTGTT | AGAGTACTGCTTGCCCAGGA |

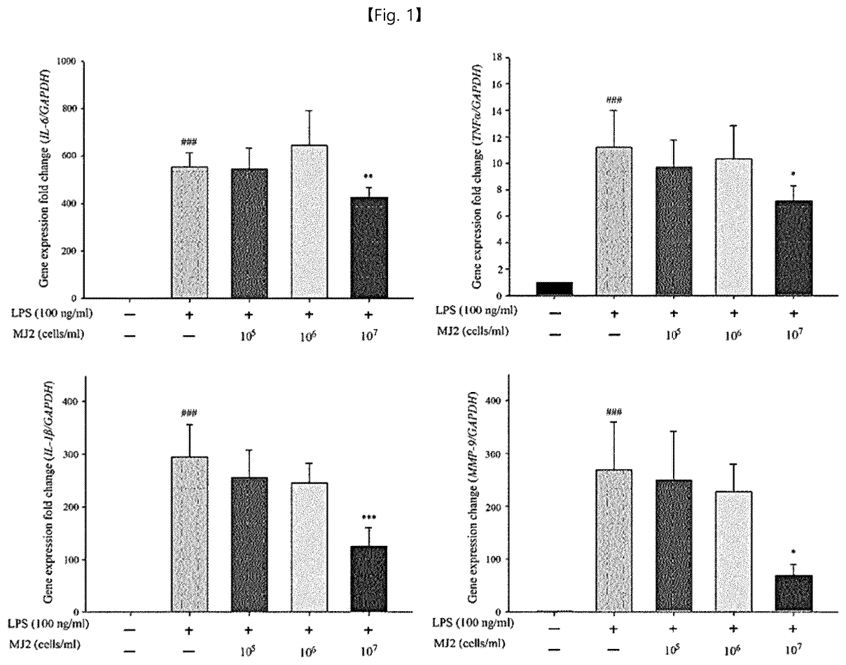

As a result, as illustrated in FIG. 1, analyzed through one-way ANOVA, in a group treated with P. freudenreichii MJ2 dead cells of 1×10⁷ cells/mL, the expression of inflammatory cytokine genes (IL-6, TNF-α, IL-1β and MMP9) was significantly reduced compared to a group treated with LPS alone.

### Example 3: Measurement of Osteoclast Differentiation Inhibitory Ability of P. Freudenreichii MJ2 Dead Cells

3-1. Culture of Osteoclasts and Treatment of P. freudenreichii MJ2 Dead Cells

A Raw 264.7 cell line, a macrophage cell line, was treated with 50 ng/mL receptor activator of nuclear factor kappa-B ligand (RANKL) for 4 days to induce differentiation into osteoclasts. The cell line was cultured in a 37° C. incubator under a 5% CO₂-95% air composition in a minimum essential medium eagle-alpha modification (α-MEM) medium added with 10% FBS, 100 units/mL penicillin, and 100 μg/mL streptomycin. When the cells grew to about 80% confluent, the cells were removed using a scraper to be adjusted to 1×10⁴ cells/mL, and then 1 mL each was inoculated into 12 wells. The cells were stabilized in a 37° C. incubator for one day, and then treated with RANKL and P. freudenreichii MJ2 dead cells (1×10⁵, 1×10⁶, and 1×10⁷ cells/mL) and cultured for 4 days.

3-2. Measurement of Osteoclast Differentiation Factor Expression Inhibitory Ability of P. freudenreichii MJ2 Dead Cells

A macrophage culture supernatant cultured by treating RANKL and MJ2 dead cells in Example 3-1 was removed, and RNA was isolated using Trizol. The isolated RNA was converted to cDNA using a revert aid first strand cDNA kit, and then subjected to real-Time PCR using an Applied Biosystems 7500 machine using a DyNamo HS SYBR Green qPCR kit reagent. The sequences of the primers used were shown in Table 2 below.

TABLE 2

| Gene | Forward (5′-3′) | Reverse (5′-3′) |
|---|---|---|
| MMP9 | GAAGGCAAACCCTGTGTGTT | AGAGTACTGCTTGCCCAGGA |
| RANK | TGCAGCTCAACAAGGATACG | GAGCTGCAGACCACATCTGA |
| c-fos | CCAGTCAAGAGCATCAGCAA | AAGTAGTGCAGCCCGGAGTA |
| NFATc1 | GGTGCTGTCTGGCCATAACT | GCGGAAAGGTGGTATCTCAA |
| Calcr | CGGACTTTGACACAGCAGAA | GTCACCCTCTGGCAGCTAAG |
| Ctsk | CAGCTTCCCCAAGATGTGAT | AGCACCAACGAGAGGAGAAA |
| OPG | CTGCCTGGGAAGAAGATCAG | TTGTGAAGCTGTGCAGGAAC |
| RANKL | AGCCGAGACTACGGCAAGTA | GCGCTCGAAAGTACAGGAAC |

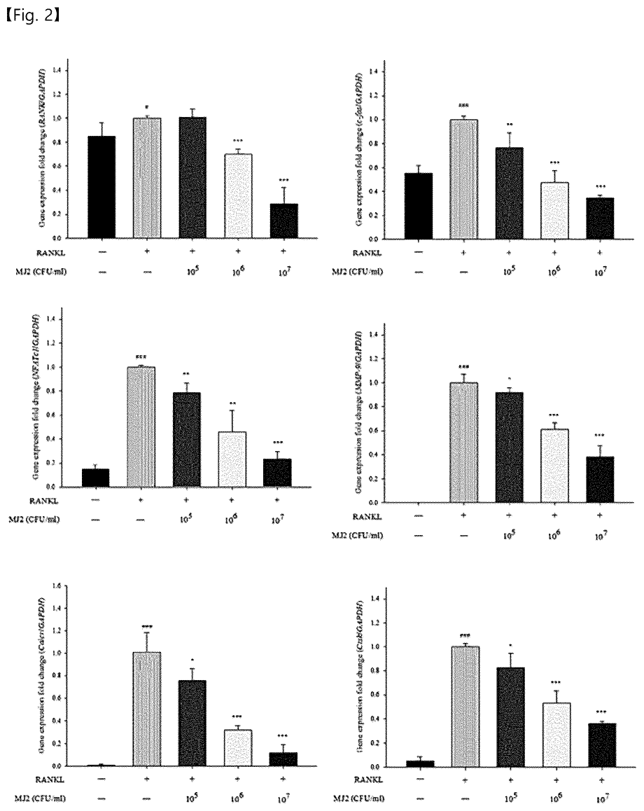

As a result, as illustrated in FIG. 2, in groups treated with P. freudenreichii MJ2 dead cells of 1×10⁶ cells/mL and 1×10⁷ cells/mL, the expression levels of genes related to osteoclast differentiation were significantly reduced compared to a group treated with RANKL alone.

3-3. Measurement of Tartrate-Resistant Acid Phosphatase (TRAP) Activity Inhibitory Ability in Osteoclasts of P. freudenreichii MJ2 Dead Cells

TRAP was an enzyme expressed during differentiation into osteoclasts, and the degree of differentiation of osteoclasts may be confirmed through TRAP staining. Then, a macrophage culture supernatant cultured by treating RANKL and MJ2 dead cells in Example 3-1 was removed, and the cells were washed with PBS and then immobilized. Sodium tartrate was added to a premixed substrate NABP/FRVLB, and then TRAP of osteoclasts was stained with the corresponding solution for 30 minutes at 37° C. The supernatant was removed, the cells were washed with distilled water, and then osteoclasts with three or more nuclei were observed under an optical microscope. To measure the TRAP activity, the immobilized cells were cultured in a citrate buffer containing sodium tartrate and para-nitrophenylphosphate (pNPP), and then the reaction of the reaction mixture was stopped with NaOH, and the absorbance was measured at 405 nm, and corrected through protein quantification.

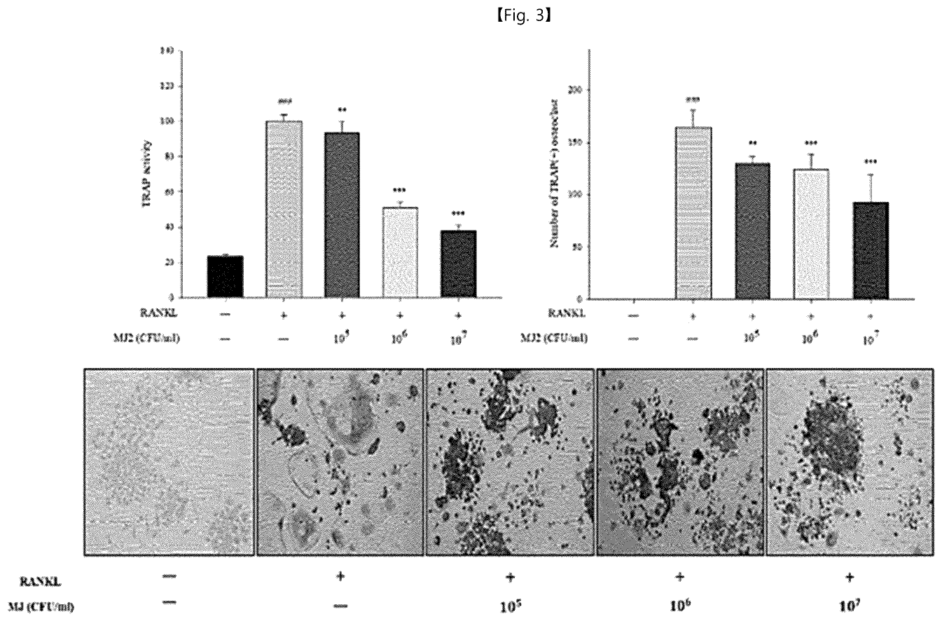

As a result, as illustrated in FIG. 3, in the group treated with P. freudenreichii MJ2 dead cells, the number of TRAP-activated osteoclasts and TRAP activity were significantly reduced compared to the group treated only with RANKL. From the result, it can be seen that osteoclast differentiation was inhibited by treatment with P. freudenreichii MJ2 dead cells.

### Example 4: Measurement of Increased Interleukin-10 (IL-10) Expression Ability of P. Freudenreichii MJ2 Dead Cells

IL-10 is a representative anti-inflammatory cytokine and is known to prevent the osteoclast differentiation by not only inhibiting inflammatory reactions, but also inhibiting the expression of nuclear factor of activated T-cells cl (NFATc1), a key factor in osteoclast differentiation. To measure the expression level of IL-10 by P. freudenreichii MJ2 dead cells, the supernatant was taken, and an ELISA method was performed.

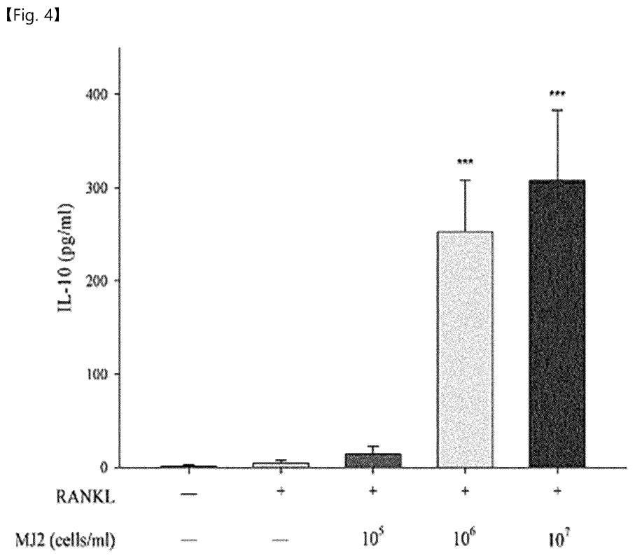

As a result, as illustrated in FIG. 4, in groups treated with P. freudenreichii MJ2 dead cells of 1×10⁶ cells/mL and 1×10⁷ cells/mL, the IL-10 expression level was significantly increased compared to a group treated with RANKL alone.

### Example 5: Evaluation of Efficacy of Preventing Rheumatoid Arthritis by P. Freudenreichii MJ2 Dead Cells

### 5-1. Evaluation of Disease Score in Collagen-Induced Arthritis Mouse Model

P. freudenreichii MJ2 was directly injected into DBA/1 mice via oral administration for 3 weeks before inducing arthritis, and arthritis was induced with collagen. Groups were divided into a group (Control) without induced arthritis after feeding PBS, a group (Vehicle) with induced arthritis after feeding PBS, a group (High-dose Live; HDL) with induced arthritis after feeding P. freudenreichii MJ2 live cells at a high dose of 1×10⁸ CFU/mL, a group (Low-dose Live; LDL) with induced arthritis after feeding P. freudenreichii MJ2 live cells at a low dose of 1×10⁷ CFU/mL, a group (High-dose Dead; HDD) with induced arthritis after feeding P. freudenreichii MJ2 dead cells at a high dose of 1×10⁸ cells/mL, and a group (Low-dose Dead; LDD) with induced arthritis after feeding P. freudenreichii MJ2 dead cells at a low dose of 1×10⁷ cells/mL. For arthritis induction, type 2 bovine collagen was emulsified with complete Freund's adjuvant (CFA) at a ratio of 1:1 and then injected into the tail dermis to induce the first immunization (1st immunization), and emulsified with incomplete Freund's adjuvant (IFA) at a ratio of 1:1 after 3 weeks and then injected into the tail dermis to induce the second immunization (2nd boost). After the arthritis was induced, the swelling symptoms and the severity of inflammation were visually evaluated according to the criteria of the arthritic score and scored.

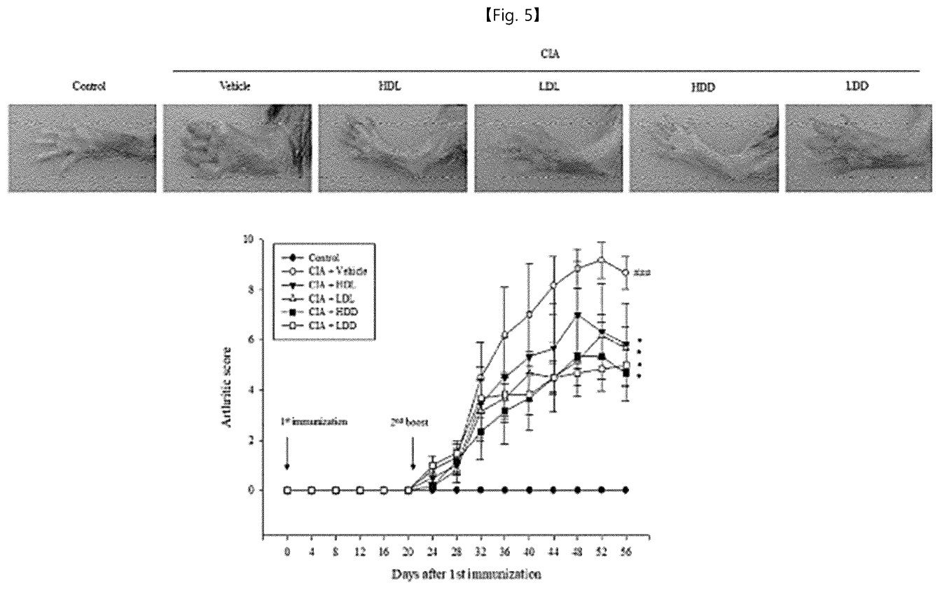

As a result, as illustrated in FIG. 5, the most severe arthritis symptoms appeared in a group with induced arthritis by administering PBS, and a group with induced arthritis by administering P. freudenreichii MJ2 live and dead cells showed a significant visual relief effect of arthritis symptoms.

### 5-2. Micro-CT Image Analysis in Collagen-Induced Arthritis Mouse Model

In addition to visual evaluation, the knee joints and paw regions of mice were photographed using micro-CT, converted to 3D images, and observed.

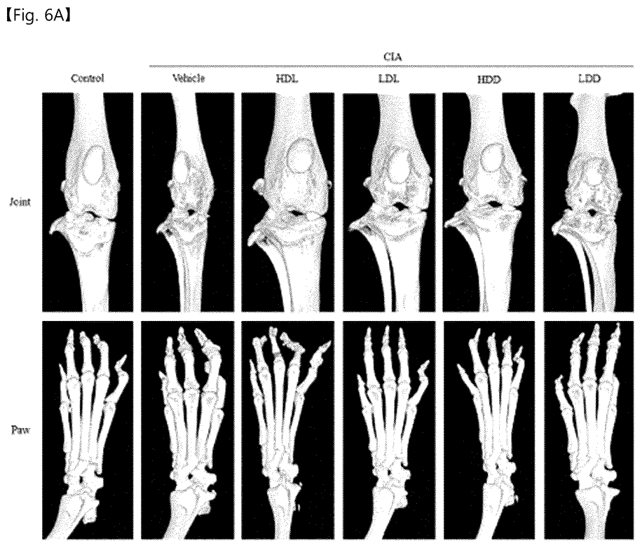

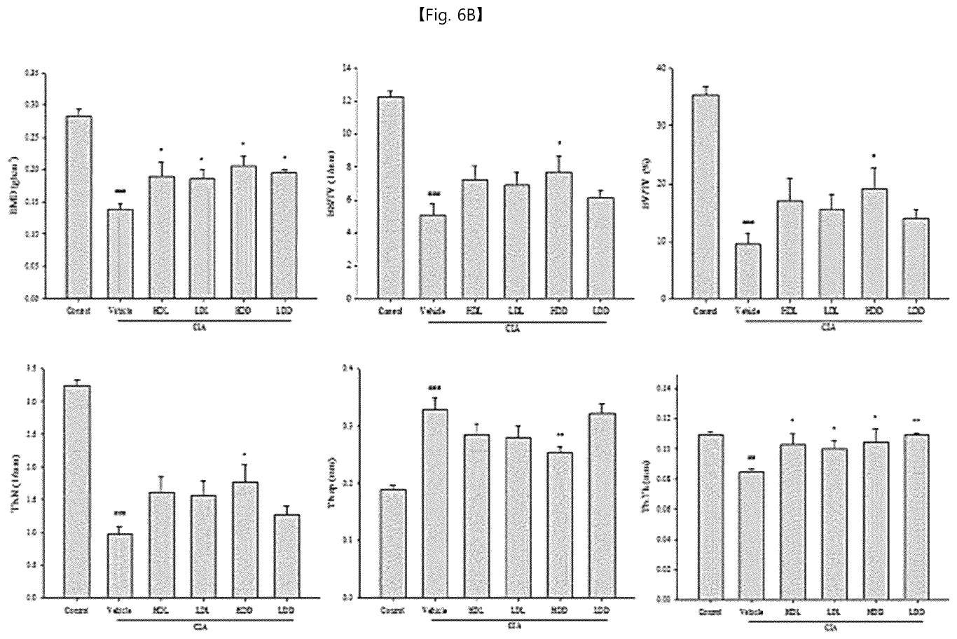

As a result, as illustrated in FIGS. 6A and 6B, there was no clear difference between groups in the paw regions, but in the knee regions, the group with induced arthritis by administering PBS had thinner bones and significant deformation and destruction of joints, and relatively, the group with induced arthritis by administering P. freudenreichii MJ2 live and dead cells showed an effect of alleviating arthritis symptoms. In scores obtained through micro-CT images, the significance was different, but the high-dose group of P. freudenreichii MJ2 dead cells showed significant results in all scores.

### 5-3. Measurement of Concentrations of Collagen Antigen-Specific IgG, IgQ1, and IgG2a Antibodies in Collagen-Induced Arthritis Mouse Model

Blood was collected from mice, plasma was separated, and concentrations of collagen antigen-specific IgG, IgG1, and IgG2a antibodies were measured using ELISA. An immunoplate was coated with mouse type II collagen at 4° C., a blocking process was performed at room temperature, and then the sample was reacted with horseradish peroxidase (HRP)-conjugated anti-mouse IgG antibody, anti-IgG1 antibody, and anti-IgG2a antibody for about 3 hours. After 3 hours, the substrate was added, and the reaction was stopped using 2 N H₂SO₄ to measure the amount of each IgG antibody in the plasma.

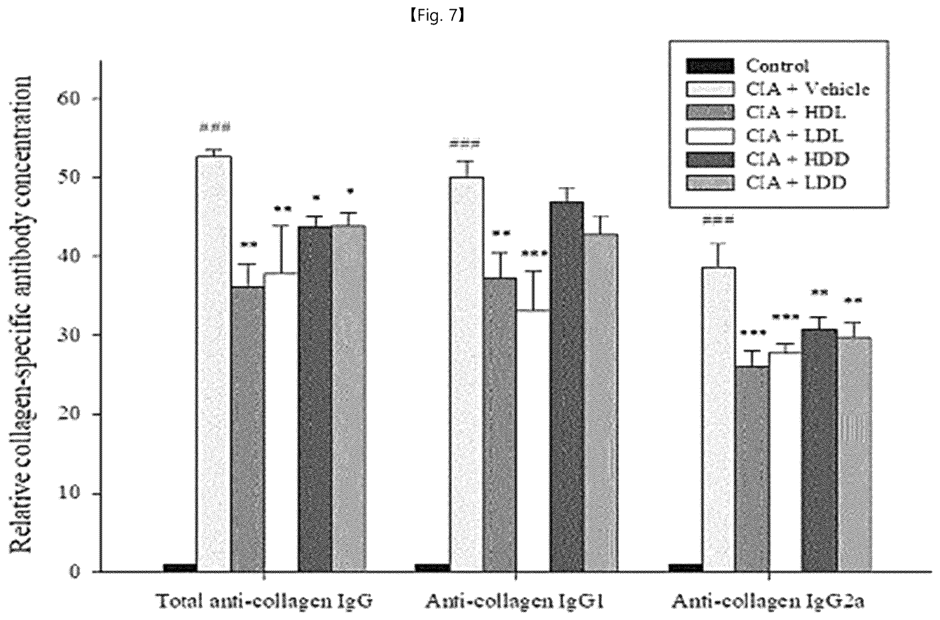

As a result, as illustrated in FIG. 7, it was confirmed that the production of collagen antigen-specific IgG antibodies in the blood was promoted when arthritis was induced, and it was confirmed that in the group administered with P. freudenreichii MJ2 live and dead cells, the concentrations of collagen antigen-specific total IgG antibody and IgG2a antibodies were decreased.

5-4. Measurement of Anti-Inflammatory Effects of P. freudenreichii MJ2 in Collagen-Induced Arthritis Mouse Model

The spleen of the mouse was separated and weighed, and then divided by the body weight of the subject to obtain the spleen index.

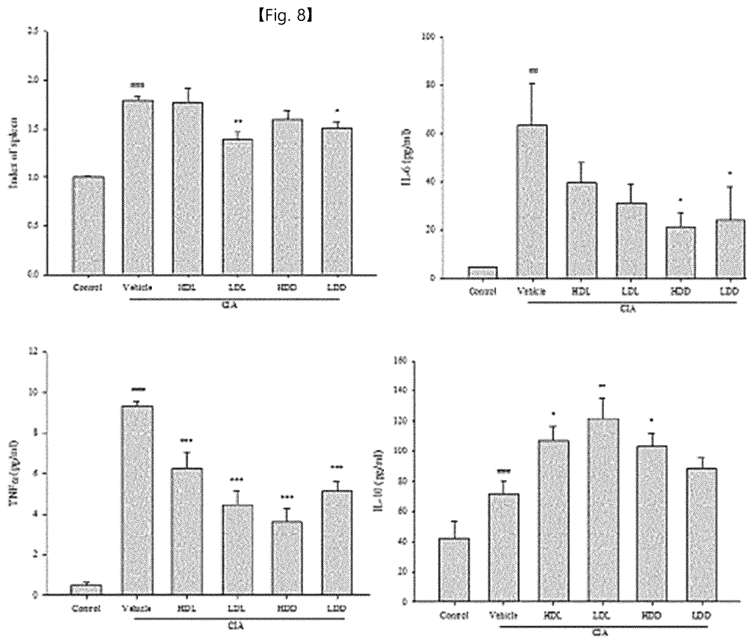

As a result, as illustrated in FIG. 8, the spleen index value was significantly high when arthritis was induced, and the spleen index values were significantly low in the P. freudenreichii MJ2 live cell low-dose and dead cell low-dose groups.

The blood was collected from mice, the plasma was separated, and the amounts of representative inflammatory cytokines, IL-6 and TNF-α, in the plasma were measured using ELISA, and the spleen of the mouse was pulverized and the amount of a representative anti-inflammatory cytokine, IL-10 was measured using ELISA.

As a result, as illustrated in FIG. 8, in the group with induced rheumatoid arthritis, the amounts of IL-6 and TNF-α were significantly increased compared with the group without induced rheumatoid arthritis. In the group administered with P. freudenreichii MJ2 dead cells, IL-6 was significantly decreased, and in the groups administered with P. freudenreichii MJ2 live and dead cells, TNF-α was significantly decreased. In all groups except for the low dose of P. freudenreichii MJ2 dead cells, IL-10 tended to increase.

5-5. Measurement of Osteoclast Differentiation Factor Expression Inhibitory Ability of P. freudenreichii MJ2 in Collagen-Induced Arthritis Mouse Model

The synovial tissue near the mouse hind limbs was pulverized, and RNA was isolated using Trizol, converted to cDNA, and subjected real-time PCR

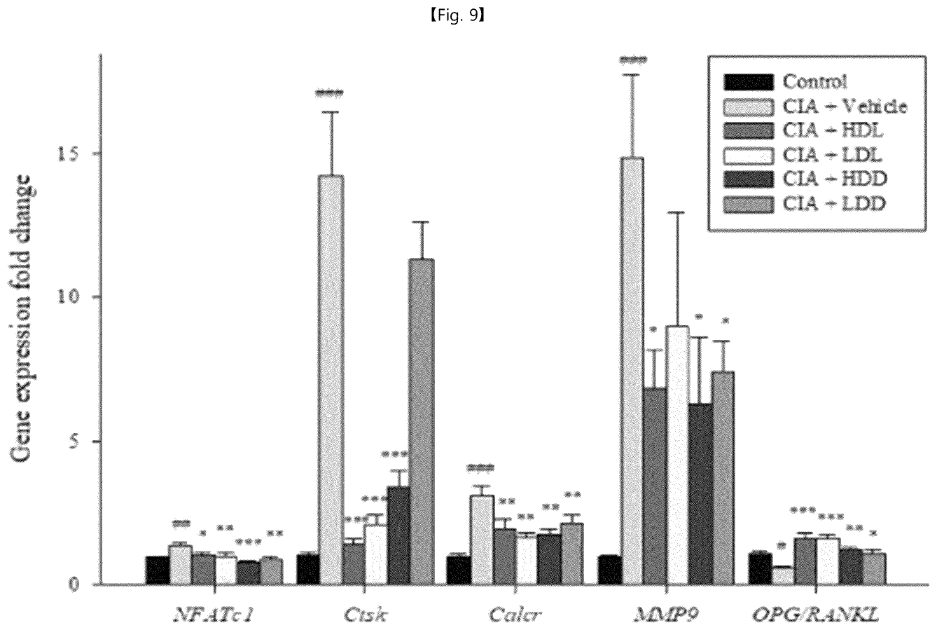

As a result, as illustrated in FIG. 9, in the group with induced rheumatoid arthritis, the expression levels of osteoclast differentiation-related genes NFATc1, cathepsin K (Ctsk), calcitonin receptor (Calcr), and matrix metallopeptidase 9 (MMP9) were significantly increased compared to the group without induced rheumatoid arthritis, and in the groups administered with P. freudenreichii MJ2, the expression levels of these genes were significantly decreased. In addition, the expression level ratio of osteoprotegerin (OPG)/RANKL, which reflects the inhibition of the differentiation of osteoclasts and the promotion of the differentiation of osteoblasts, was significantly increased in the groups administered with P. freudenreichii MJ2.

5-6. Measurement of Apoptosis Inhibitory Ability of P. freudenreichii MJ2 in Collagen-Induced Arthritis Mouse Model

The synovial tissue near the mouse hind limbs was pulverized, the amount of protein was quantified using a Bradford method, and denatured at 100° C., and western blot was performed.

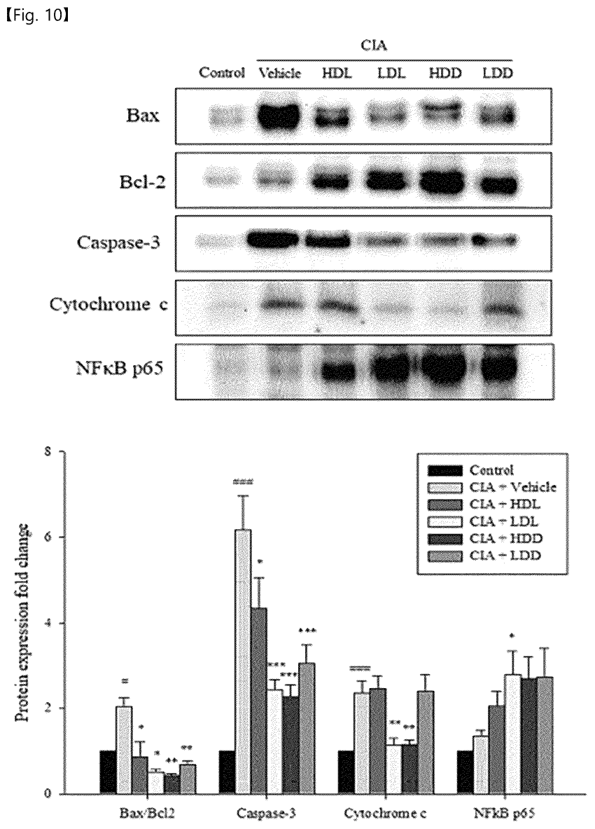

As a result, as illustrated in FIG. 10, in the group with induced rheumatoid arthritis, the expression levels of Bax/Bcl2 ratio, caspase-3 and cytochrome c were significantly increased compared to the group without induced rheumatoid arthritis, and in the groups administered with P. freudenreichii MJ2, the expression levels of these proteins were significantly decreased.

5-7. Measurement of Autophagy Expression of P. freudenreichii MJ2 in Collagen-Induced Arthritis Mouse Model

The synovial tissue near the mouse hind limbs was pulverized, the amount of protein was quantified using a Bradford method, and denatured at 100° C., and the expression level of LC3B, a representative factor indicating autophagy expression, was measured through western blot.

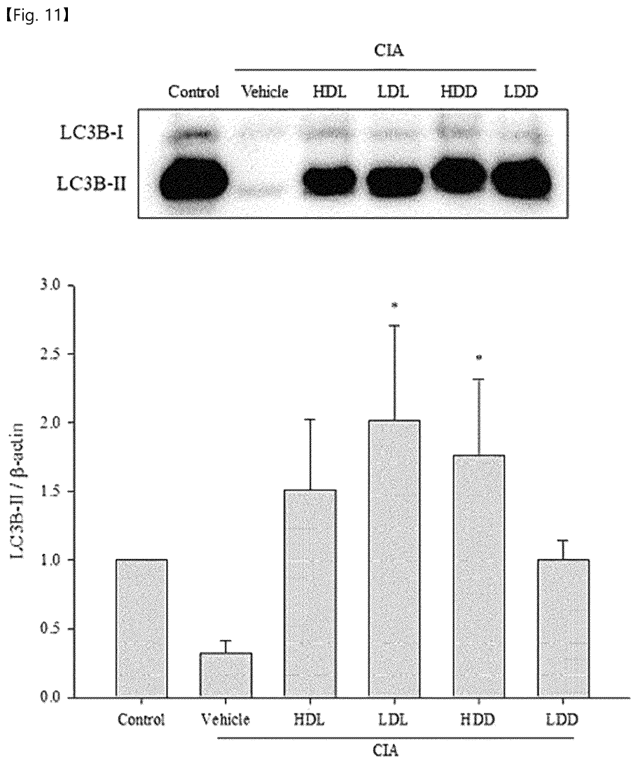

As a result, as illustrated in FIG. 11, in the group with induced rheumatoid arthritis, the expression level of LC3B was significantly decreased compared to the group without induced rheumatoid arthritis, and in the groups administered with P. freudenreichii MJ2, the expression level of LC3B was significantly increased.

As described above, specific parts of the present invention have been described in detail, and it will be apparent to those skilled in the art that these specific descriptions are merely preferred embodiments, and the scope of the present invention is not limited thereto. Therefore, the substantial scope of the present invention will be defined by the appended claims and their equivalents.

1. A pharmaceutical composition for treating rheumatoid arthritis comprising Propionibacterium freudenreichii MJ2 (KCCM12272P), a culture solution thereof, live cells thereof, dead cells thereof, or a mixture thereof as an active ingredient.

2. The pharmaceutical composition of claim 1, wherein the live cells are obtained by culturing the MJ2 strain activated in a culture medium for 45 to 50 hours under anaerobic conditions at 30 to 37° C. and centrifuging the cultured strain solution.

3. The pharmaceutical composition of claim 1, wherein the dead cells are obtained by culturing the MJ2 strain activated in a culture medium for 45 to 50 hours under anaerobic conditions at 30 to 37° C. and heat-treating the cultured strain.

4. The pharmaceutical composition of claim 1, wherein the composition is for oral administration.

5. The pharmaceutical composition of claim 1, wherein the composition inhibits inflammatory reactions by increasing expression of IL-10.

6. The pharmaceutical composition of claim 1, wherein the composition inhibits osteoclast differentiation.

7. The pharmaceutical composition of claim 1, wherein the composition decreases production of IgG, IgG1, and IgG2a.

8. The pharmaceutical composition of claim 1, wherein the composition inhibits apoptosis and restores autophagy to a normal level.

9. The pharmaceutical composition of claim 1, wherein the composition includes the live cells at a concentration of 1×10⁶ to 1×10¹⁰ CFU/mL, and the dead cells at a concentration of 1×10⁶ to 1×10¹⁰ cells/mL.

10. A food composition for alleviating rheumatoid arthritis comprising Propionibacterium freudenreichii MJ2 (KCCM12272P), a culture solution thereof, live cells thereof, dead cells thereof, or a mixture thereof as an active ingredient.

11. The food composition of claim 10, wherein the food is a functional food, and
the composition includes the live cells at a concentration of 1×10⁶ to 1×10¹⁰ CFU/mL, and the dead cells at a concentration of 1×10⁶ to 1×10¹⁰ cells/mL.

12. A method for treating rheumatoid arthritis, the method comprising administering the pharmaceutical composition according to claim 1, the culture solution thereof, live cells thereof, dead cells thereof, or the mixture thereof as an active ingredient to a subject in need thereof.
